(19) United States
(12) Patent Application Publication
(10) Pub. No.: US 2025/0223615 A1
(43) Pub. Date: Jul. 10, 2025

(54) METHOD FOR PREPARING A COMPOUND OF FORMULA (I) BY FERMENTATION

(71) Applicant: SPECIALTY OPERATIONS FRANCE, Lyon (FR)

(72) Inventors: Laurent Garel, Lyon (FR); Guillaume Pireau, Lyon (FR); Chenglu David, Lyon (FR)

(73) Assignee: SPECIALTY OPERATIONS FRANCE, Lyon (FR)

(21) Appl. No.: 18/854,943

(22) PCT Filed: Apr. 14, 2023
(86) PCT No.: PCT/EP2023/059802

§ 371 Date: Oct. 7, 2024

(30) Foreign Application Priority Data
Apr. 15, 2022 (FR) 2203535

(51) Int. Cl. C12P 7/26

(52) U.S. Cl. CPC C12P 7/26

(57) ABSTRACT

The present invention relates to a method for preparing a compound of formula (I) in which n is between 0 and 5, preferably between 1 and 2; R, which can be identical or different, is chosen from the group made up of OH, OR₁, formyl, CO₂H, linear or branched alkyl chains, which are optionally substituted, comprising between 1 and 6 carbon atoms, alkenyls, which may be linear or branched and optionally substituted, comprising between 1 and 6 carbon atoms; R1 is chosen from the group made up of methyl, ethyl, propyl and butyl, comprising a step (a) in which a substrate is transformed into a compound of formula (I) by bioconversion in the presence of a microorganism, which method is characterised in that step (a) is performed under non-aseptic, preferably non-sterile, conditions.

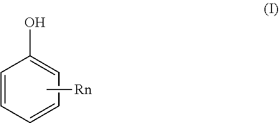

### FIELD OF THE INVENTION

The present invention relates to a process for preparing a compound of formula (I), comprising a step (a) wherein a substrate is converted into a compound of formula (I) by bioconversion in the presence of a microorganism, characterized in that step (a) is carried out under non-aseptic conditions, preferably under non-sterile conditions.

### PRIOR ART

Vanillin may be obtained by various methods known to a person skilled in the art, and notably by the following two routes:
a “natural” route based on a biotechnological process notably comprising the cultivation of a microorganism that is capable of enabling the bioconversion of a fermentation substrate into vanillin. Such a process, wherein the fermentation substrate is ferulic acid, is notably known from patent application EP 0885968. U.S. Pat. No. 5,017,388 describes a process wherein the fermentation substrate is eugenol and/or isoeugenol. These processes lead to the preparation of a vanillin known as natural vanillin.
a “synthetic” route comprising conventional chemical reactions starting from guaiacol and not involving microorganisms. This process leads to the preparation of a vanillin known as synthetic vanillin.

Finally, vanillin may also be prepared via a “biobased” route, wherein the vanillin is derived from lignin. In particular, mention may be made of documents U.S. Pat. No. 2,745,796, DE 1132113 and the article entitled “Preparation of lignin from wood dust as vanillin source and comparison of different extraction methods” by Azadbakht et al. in International Journal of Biology and Biotechnology, 2004, vol. 1, No. 4, pages 535-537, or prepared from materials of natural origin. Mention may particularly be made of document WO 2019/020773.

Frambinone may also be obtained by various methods and in particular by a bioconversion process as described in document CN 112391418.

The processes enabling the production of a vanillin or a frambinone referred to as “natural” are carried out in fermentors, under sterile conditions. Sterilization is generally achieved by injecting steam into the devices used for a period generally exceeding 20 min. The reagents are generally also sterile, in particular by virtue of having been passed through a filtering membrane. These processes are therefore tedious and expensive to implement and require specific equipment that may be sterilized; the energy impact of these processes is also very high.

The present invention aims to provide a process for preparing a compound of formula (I) which is simple to implement and the ecological and/or energy footprint of which is improved.

### BRIEF DESCRIPTION

A first subject of the present invention relates to a process for preparing a compound of formula (I), comprising a step (a) wherein a substrate is converted into a compound of formula (I) by bioconversion in the presence of a microorganism, characterized in that step (a) is carried out under non-sterile conditions.

### DETAILED DESCRIPTION

In the context of the present invention, and unless otherwise indicated, the expression “between . . . and . . . ” includes the limits.

In the context of the present invention, and unless otherwise indicated, the growth of a microorganism refers to a process wherein said microorganism multiplies. Growth leads to the production of biomass.

In the context of the present invention, and unless otherwise indicated, the term “bioconversion” refers to a biotechnological process wherein a microorganism allows the conversion of a substrate into a bioconversion product. The microorganism may be a genetically modified wild-type strain that has been obtained by molecular biology or mutated, in particular by random or site-directed mutagenesis.

In the context of the present invention, and unless otherwise indicated, the term “process carried out under aseptic conditions” refers to a process carried out under conditions free of any microbe and/or microorganism.

In the context of the present invention, and unless otherwise indicated, the term “process carried out under sterile conditions” refers to a process carried out under conditions free of any microbe and/or microorganism liable to be detrimental to the microorganism capable of converting a substrate into a compound of formula (I) used in the context of the process of the present invention. A detrimental effect on the microorganism capable of converting a substrate into a compound of formula (I) may be measured by the degree of conversion of said substrate into a compound of formula (I).

In the context of the present invention, and unless otherwise indicated, the asepsis or sterilization may be obtained by any method, in particular thermal, chemical, mechanical or radiation method, enabling the elimination of microbes and/or microorganisms. The asepsis or the sterilization may be performed on the fluids, starting materials, and/or devices used in the process according to the present invention. By way of illustration, mention may be made in particular of the following methods:
Wet- or dry-heat sterilization, carried out by raising the temperature to a sufficient temperature, followed by maintaining this temperature for a sufficient period of time to eliminate the microbes and/or microorganisms; by way of illustration, the temperature may be 121° C., followed by maintaining this temperature of 121° C. for a sufficient period of time to eliminate the microbes,
Membrane filtration or several successive membrane filtrations, the smallest cut-off diameter of which allows the removal of any microbe and/or microorganism.

In the context of the present invention, and unless otherwise indicated, the term “reactor” refers to a vessel suitable for carrying out chemical reactions. In the context of the present invention, the reactor may be used after having been made aseptic or sterilized or without aseptization or sterilization.

In the context of the present invention, and unless otherwise indicated, the term “fermentation” refers to any biological reaction involving a microorganism, such as bacterial growth, bioconversion, biosynthesis or biocatalysis.

In the context of the present invention, and unless otherwise indicated, the term “fermentor” refers to a reactor suitable for carrying out fermentations, for example a bioreactor. In the context of the present invention, the fermentor may be used after having been made aseptic or sterilized or without aseptization or sterilization.

In the context of the present invention and unless otherwise indicated, a compound of formula (I) obtained by a bioconversion process may be termed a natural compound of formula (I).

In the context of the present invention and unless otherwise indicated, a frambinone obtained by a bioconversion process may be termed a natural frambinone.

In the context of the present invention and unless otherwise indicated, a vanillin obtained by a bioconversion process may be termed a natural vanillin.

A first aspect of the present invention relates to a process for preparing a compound of formula (I), comprising a step (a) wherein a substrate is converted into a compound of formula (I) by bioconversion in the presence of a microorganism, characterized in that step (a) is carried out under non-sterile conditions.

In the context of the present invention, a compound of formula (I) has the following formula:

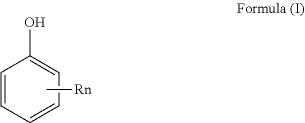

wherein n is between 0 and 5, preferably between 1 and 2,
R, which may be identical or different, is chosen from the group consisting of OH, OR₁, formyl, CO₂H, optionally substituted linear or branched alkyl chains comprising between 1 and 6 carbon atoms, optionally substituted linear or branched alkenyl chains comprising between 1 and 6 carbon atoms.

According to a particular embodiment, R₁ is chosen from the group consisting of methyl, ethyl, propyl and butyl.

According to a particular embodiment, R is chosen from the group consisting of formyl —CHO, OH, OMe, OEt, OPr, OBu, methyl, ethyl, —(CH₂)₂—C(O)—CH₃, —CH═CH—C(O)—CH₃, —CH═CH—CO₂H, —CO₂H, —CH₂—OH.

According to a particular embodiment, the compound of formula (I) is chosen from vanillin and frambinone.

In the context of the present invention, the microorganism may be any microorganism capable of forming the compound of formula (I) by bioconversion of an appropriate substrate. The microorganism, in particular bacterium or fungus, may be a wild-type microorganism that has been genetically modified by molecular biology, or mutated, by random or site-directed mutagenesis.

Preferably, the microorganism is chosen from bacteria belonging to the order Actinomycetales, Caulobacterales or Pseudomonadales, preferably belonging to the family Streptomycetacae, Pseudonocardiaceae, Micrococcaceae, Caulobacteraceae or Pseudomonadaceae, very preferentially Streptomyces setonii, Amycolatopsis sp., Streptomyces psammoticus, Amycolatopsis thermoflava, Micrococcus sp., Caulobacter segnis, Pseudomonas fluorescens, or mutants thereof, even more preferentially Streptomyces setonii, Amycolatopsis sp., Streptomyces psammoticus. Preferably, the bioconversion reaction is carried out in the presence of a strain available under the number ATCC39116, DSMZ 9991, DSMZ 9992, CCTCC 2015329, CCTCC 2011265, IMI 390106 or Zyl 926 or mutants thereof, preferentially ATCC39116, DSMZ 9991, DSMZ 9992, CCTCC 2015329, IMI 390106 or Zyl 926 or mutants thereof and very preferentially ATCC39116, DSMZ 9991, DSMZ 9992, CCTCC 2015329, IMI 390106 or Zyl 926. According to another embodiment, the microorganism may be chosen from genetically modified microorganisms belonging to the family Saccharomyces cerevisiae, Schizosaccharomyces pombe, E. coli, Corynebacterium glutamicum.

The microorganism used in the context of the present invention may also be a strain as described in WO 2014/102368, WO 2016/001203 or EP 2721148.

According to another embodiment, the microorganism is chosen from fungi belonging to the genus Aspergillus, Pycnoporus, Penicillium, preferably belonging to the family Aspergillus luchuensis, Aspergillus niger, Pycnoporus cinnabarinus, Penicillium camemberti.

In the context of the present invention, the substrate may be converted by bioconversion into a compound of formula (I).

In the context of the present invention, the substrate may be converted by bioconversion into frambinone. The substrate is generally chosen from the group consisting of p-coumaric acid and p-hydroxybenzalacetone.

In the context of the present invention, the substrate may be converted by bioconversion into vanillin. The substrate is generally chosen from the group consisting of glucose, ferulic acid, eugenol, isoeugenol, coumaric acid, caffeic acid, L-tyrosine, vanillic acid and 4-vinylguaiacol, preferably chosen from glucose, ferulic acid and eugenol.

In the context of the present invention, step (a) of the process is carried out under non-aseptic, preferably non-sterile, conditions. In the context of the present invention, the expression “step (a) is carried out under non-aseptic, preferably non-sterile, conditions” indicates that the reactor or fermentor in which step (a) is carried out, and optionally the various feed or outlet pipes of the reactor or fermentor, have not been rendered aseptic, preferably have not been sterilized, prior to their use in step (a). In the context of the present invention, the expression “step (a) is carried out under non-aseptic, preferably non-sterile, conditions” indicates that the fermentation substrate and/or the solvent used in step (a) have not been rendered aseptic, preferably have not been sterilized, prior to their use in step (a). In the context of the present invention, the expression “step (a) is carried out under non-aseptic, preferably non-sterile, conditions” indicates that the transfer of the biomass used for fermentation is not carried out aseptically, preferably is not carried out sterilely. In the context of the present invention, the expression “step (a) is carried out under non-aseptic, preferably non-sterile, conditions” indicates that the gas or gases, in particular air or oxygen, used during step (a) have not been rendered aseptic, preferably have not been sterilized, prior to their use in step (a).

The absence of aseptization, preferably of sterilization, of the equipment, reagents and air or oxygen used during the bioconversion step (a) is particularly advantageous, in particular in that it allows the use of simpler equipment. The energy cost of the bioconversion process is also improved. Indeed, the absence of aseptization, preferably sterilization, reduces steam consumption and reduces the carbon footprint of the process.

In the context of the present invention, step (a) may be carried out batchwise or in fed-batch mode. According to another embodiment, step (a) may be carried out continuously. Step (a) may also be carried out in a cascade of perfectly stirred reactors in series.

According to a particular embodiment, the fermentation substrate is introduced into the reactor or fermentor. A biomass is then introduced into the reactor.

According to another particular embodiment, the fermentation substrate is introduced into the reactor containing the biomass.

Advantageously, the total concentration of fermentation substrate and of compound of formula (I) is between 0.1 g/l and 50 g/1. According to a particular embodiment, the total concentration of fermentation substrate and of compound of formula (I) is between 0.1 g/l and 25 g/1, preferably between 0.2 g/l and 20 g/1. According to another particular embodiment, the total concentration of fermentation substrate and of compound of formula (I) is between 10 g/l and 50 g/1, preferably between 12 g/l and 40 g/1, very preferentially between 15 g/l and 35 g/1.

The pH during step (a) is controlled and is between 7 and 9. The pH may optionally be adjusted by adding base or acid.

Step (a) is generally carried out at a temperature of between 25 and 50° C., preferably between 28° C. and 45° C. Step (a) is generally carried out with stirring. Step (a) is generally carried out under air or oxygen. In the context of the present invention, the pO2 during step (a) is controlled and optionally adjusted. A person skilled in the art will be able to adjust the pO2 during step (a) to the needs of the microorganism capable of converting a substrate into a compound of formula (I), preferably into vanillin or frambinone, very preferentially into vanillin, used in the context of the present invention. By way of illustration, the pO2 may be between 1% and 99%, preferably of the order of 10%, 20%, 30%, 40%, 50%, 60%, 70%, 80%, 90%. The pO2 corresponds to the dissolved oxygen concentration in the bioconversion medium relative to the dissolved oxygen concentration at saturation, without biomass. The pO2 may in particular be measured with a Hamilton Oxyferm FDA VP 225 O₂ sensor.

Advantageously, the process of the invention is not only more sustainable, due to the reduction in its energy consumption, while making it possible to obtain a yield of compound of formula (I) at least equivalent to those of the processes requiring asepsis or sterilization.

In general, the bioconversion step (a) is preceded by a step (a0) during which a microorganism is cultured so as to obtain a sufficient amount of biomass. This biomass may then be used in a bioconversion process according to the present invention.

The culturing of the bacterium is generally performed in an aqueous medium, in the presence of nutrient elements. In general, the culture medium comprises a carbon source, an organic or inorganic nitrogen source, inorganic salts and growth factors. A person skilled in the art will be able to adjust the composition of the culture medium to the needs of the microorganism capable of converting a substrate into a compound of formula (I) used in the context of the process of the present invention.

The concentration of the carbon source is generally between 5 and 50 g·l⁻¹, preferably between 20 and 34 g·l⁻¹. The nitrogen source, such as a yeast extract, and the growth factors are generally added at a concentration of between 2 and 20 g·l⁻¹, preferably between 5 and 10 g·l⁻¹. In addition, magnesium ions, such as magnesium sulfate, may be added at a concentration of between 0.1 and 5 g·l⁻¹, preferably between 0.5 and 1 g·l⁻¹. In general, the pH of the culturing is between 3 and 9, in particular between 4 and 8, and very preferentially between 6 and 8. In general, the temperature of the culturing is between 1° and 55° C., particularly preferably within the range of 30 to 50° C. and very preferentially between 35° C. and 45° C.

The culture period generally lasts between 15 minutes and 80 hours, preferably between 1 hour and 50 hours and very preferentially between 5 hours and 40 hours. The duration of the culture period is variable and may be adjusted as a function of the microorganism capable of converting a substrate into a compound of formula (I) used in the context of the present invention. By way of illustration, the culture period may last until the carbon source, generally glucose, is almost entirely consumed, preferably such that the concentration of carbon source is less than or equal to 15 g·l⁻¹, preferably less than or equal to 5 g·l⁻¹, very preferentially less than or equal to 3 g·l⁻¹. According to another embodiment, the culture period may last until 10%, 20%, 30%, 40%, 50%, 60%, 70%, 80%, 90%, 95% of the carbon source introduced at the beginning of step (a0) has been consumed.

Step (a0) is generally carried out in a fermentor, under aseptic, preferably sterile, conditions.

In the context of the present invention, step (a) may be followed by a step (b) of purifying the compound of formula (I) obtained. In general, the purification comprises a first step in which the fermentation must is separated from the biomass.

The purification of the compound of formula (I) obtained at the end of step (a) may in particular be carried out by liquid/liquid extraction, by distillation, by crystallization, by evaporation in a wiped film evaporator, a falling film evaporator, in a partition evaporator, and/or by stripping. Nanofiltration and ultrafiltration steps may also be carried out as part of the purification step (b). The purification of the vanillin obtained at the end of step (a) may in particular be carried out by liquid/liquid extraction, by distillation, by crystallization, by evaporation in a wiped film evaporator, a falling film evaporator, in a partition evaporator, and/or by stripping. Nanofiltration and ultrafiltration steps may also be carried out as part of the purification step (b). By way of illustration, the purification process may be carried out according to the processes described in WO2013/087795, WO2014/114590, WO2018/146210, WO2021/019005.

According to another embodiment, the vanillin may be extracted continuously from the fermentor by means of a vanillin separation device. By way of illustration, the purification process may be carried out according to the process described in WO2017/025339. There is no particular limitation with regard to the choice of the vanillin separation device. Preferably, the vanillin separation device is a membrane filtration device. This type of device makes it possible to recover from fermentation must, a retentate containing the microorganism and a filtrate free of microorganism. The vanillin separation device may also allow the selective extraction of the vanillin from the extracted fermentation must.

The present invention also relates to a compound of formula (I), preferably a vanillin or a frambinone, which may be obtained according to the process of the present invention. Finally, the present invention refers to the use of the compound of formula (I), preferably vanillin or frambinone, obtained according to the present invention, as a flavor in the field of human and animal food, or pharmacy, or as a fragrance in the cosmetics industry, perfumery and detergency.

### EXAMPLES

### Example 1: Culturing

The Streptomyces setonii ATCC 39116 strain is cultured under the conditions described in document WO2017/025339.

### Examples 2 and 3: Bioconversion Process According to the Invention and Comparative Process

Biomass-containing growth medium prepared according to example 1 is used.

The pH of the growth medium is adjusted to 8.4 by adding 30 mol % sodium hydroxide and kept constant, with stirring and aeration.

A solution containing ferulic acid, 30 mol % sodium hydroxide and water is prepared. This solution is then added to the fermentor or reactor containing the biomass.

### Conditions

TABLE 1

| | Example 2 - Comparative - | Example 3 according |
|---|---|---|
| | (according to EP0885968) | to the invention |
| Type of device for | Fermentor | Reactor |
| step (a) | | |
| Sterilization of the | YES | NO |
| device for step (a) | | |
| Sterilization of the | YES | NO |
| substrate | | |
| Sterilization of air | YES | NO |

The following bioconversion results were obtained:

TABLE 2

| | | Example 3 |
|---|---|---|
| | Example 2 - Comparative - | according to |
| | (according to EP0885968) | the invention |
| Vanillin | 13.9 | 15.4 |
| concentration (g/l) | | |
| Substrate conversion | 99.0 | 99.6 |
| rate (%) | | |
| % reduction in steam | — | 100% |
| consumption compared | | |
| to example 2 | | |

### Examples 4 and 5: Bioconversion Process According to the Invention and Comparative Process

Biomass-containing growth medium prepared according to example 1 is used.

The pH of the medium is adjusted to 8.4 and a coumaric acid solution is introduced so as to obtain a final concentration of between 5 and 50 g/l. The reaction medium is maintained at 37° C. and 170 rpm for 24 h. After 24 h of bioconversion, the biomass is removed by centrifugation, and the supernatant is filtered off and analyzed by HPLC. The 4-hydroxybenzaldehyde is obtained in the same proportions according to examples 4 and 5.

### Conditions

TABLE 3

| | | Example 5 according |
|---|---|---|
| | Example 4 - Comparative | to the invention |
| Type of device for | Fermentor | Reactor |
| step (a) | | |
| Sterilization of the | YES | NO |
| device for step (a) | | |
| Sterilization of the | YES | NO |
| substrate | | |
| Sterilization of air | YES | NO |

### Examples 6 and 7: Bioconversion Process According to the Invention and Comparative Process

Biomass-containing growth medium prepared according to example 1 is used. 4-Hydroxybenzalacetone is mixed in the medium obtained after the growth period as described in example 1. The mixture is incubated at 30° C. with stirring (200 rpm) for 72 hours.

After 72 h of bioconversion, the biomass is removed by centrifugation, and the supernatant is filtered off and analyzed by HPLC. The frambinone is obtained in the same proportions according to examples 6 and 7.

### Conditions

TABLE 4

| | | Example 7 according |
|---|---|---|
| | Example 6 - Comparative | to the invention |
| Type of device for | Fermentor | Reactor |
| step (a) | | |
| Sterilization of the | YES | NO |
| device for step (a) | | |
| Sterilization of the | YES | NO |
| substrate | | |
| Sterilization of air | YES | NO |

Thus, the process according to the present invention makes it possible to obtain results at least as good as the process according to comparative example 2 in terms of vanillin concentration or comparative examples 4 and 6 for the preparation of 4-hydroxybenzaldehyde and frambinone.

The results of table 2 demonstrate that the process according to the present invention is particularly advantageous in that it makes it possible in particular to reduce the steam consumption of the process and simplifies the handling by requiring no sterilization of the materials involved in the process. These results are obtained while maintaining the yields of the bioconversion step (a).

1. A process for preparing a compound of formula (I), the method comprising:
a step (a) of converting a substrate into a compound of formula (I) by bioconversion in the presence of a microorganism, wherein step (a) is carried out under non-aseptic conditions, and wherein the compound of formula (I) is 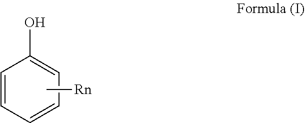
wherein n is between 0 and 5,
R, which may be identical or different, is selected from the group consisting of OH, OR₁, formyl, CO₂H, optionally substituted linear or branched alkyl chains comprising between 1 and 6 carbon atoms, optionally substituted linear or branched alkenyl chains comprising between 1 and 6 carbon atoms, and combinations thereof, wherein R₁ is selected from the group consisting of methyl, ethyl, propyl and butyl.

2. The process of claim 1, wherein step (a) is carried out under non-sterile conditions.

3. The process for preparing a compound of formula (I) as claimed in claim 1, wherein a reactor or fermentor in which step (a) is carried out, and optionally various feed or outlet pipes of the reactor or fermentor, have not been rendered aseptic prior to their use in step (a).

4. The process for preparing a compound of formula (I) as claimed in claim 1, wherein a fermentation substrate and/or a solvent used during step (a) have not been rendered aseptic prior to their use in step (a).

5. The process for preparing a compound of formula (I) as claimed in claim 1, wherein transfer of a biomass used for fermentation is not carried out under aseptic conditions.

6. The process for preparing a compound of formula (I) as claimed in claim 1, wherein a total concentration of fermentation substrate and of the compound of formula (I) is between 0.1 g/l and 50 g/l.

7. The process for preparing the compound of formula (I) as claimed in claim 1, characterized in that the gas or gases used during step (a) have not been rendered aseptic prior to their use in step (a).

8. The process for preparing the compound of formula (I) as claimed in claim 1, wherein a fermentation substrate is introduced into a reactor or fermentor, and then a biomass is introduced into the reactor or fermentor.

9. The process for preparing the compound of formula (I) as claimed in claim 1, wherein a fermentation substrate is introduced into a reactor containing a biomass.

10. The process for preparing the compound of formula (I) as claimed in claim 1, further comprising a step (a0) of culturing a microorganism so as to obtain a sufficient amount of biomass prior to step (a).

11. The process for preparing the compound of formula (I) as claimed in claim 1, further comprising a step (b) of purifying the compound of formula (I) obtained at the end of step (a).

12. The process for preparing the compound of formula (I) as claimed in claim 1, wherein the compound of formula (I) is selected from the group consisting of vanillin, frambinone, and combinations thereof.

13. The process for preparing the compound of formula (I) as claimed in claim 1, wherein n is between 1 and 2.

14. The process for preparing the compound of formula (I) as claimed in claim 3, wherein the reactor or fermentor, and optionally the various feed or outlet pipes of the reactor or fermentor, are non-sterile.

15. The process for preparing the compound of formula (I) as claimed in claim 4, wherein the fermentation substrate and/or the solvent have not been sterilized prior to their use in step (a).

16. The process for preparing the compound of formula (I) as claimed in claim 5, wherein the transfer of the biomass used for fermentation is carried out under non-sterile conditions.

17. The process for preparing the compound of formula (I) as claimed in claim 7, wherein the gas or gases are air or oxygen.

18. The process for preparing the compound of formula (I) as claimed in claim 7, wherein the gas or gases have not been sterilized prior to their use in step (a).
